(19) United States
(12) Patent Application Publication
(10) Pub. No.: US 2017/0035694 A1
(43) Pub. Date: Feb. 9, 2017

(54) THERAPEUTIC POLYMERIC NANOPARTICLES COMPRISING CORTICOSTEROIDS AND METHODS OF MAKING AND USING SAME

(71) Applicant: Pfizer Inc., New York, NY (US)

(72) Inventors: Abhimanyu Sabnis, Arlington, MA (US); Greg Troiano, Pembroke, MA (US)

(21) Appl. No.: 15/243,212

(22) Filed: Aug. 22, 2016

Related U.S. Application Data
(63) Continuation of application No. 13/523,034, filed on Jun. 14, 2012, now abandoned.
Application No. 13/523,034 is a continuation of application No. PCT/US10/60570, filed on Dec. 15, 2010.
(60) Provisional application No. 61/405,778, filed on Oct. 22, 2010.
(60) Provisional application No. 61/286,831, filed on Dec. 16, 2009.
(60) Provisional application No. 61/286,559, filed on Dec. 15, 2009.

(51) Int. Cl. A61K 9/16; A61K 31/573; C08G 63/08; A61K 31/58

(52) U.S. Cl. CPC A61K 9/1647; A61K 31/58; A61K 31/573; C08G 63/08

(57) ABSTRACT

The present disclosure generally relates to therapeutic nanoparticles. Exemplary nanoparticles disclosed herein may include about 0.1 to about 50 weight percent of a corticosteroid; and about 50 to about 99 weight percent biocompatible polymer.

### RELATED APPLICATIONS

This application is a continuation of U.S. Ser. No. 13/523,034 filed Jun. 14, 2012, which is a continuation of PCT/US10/60570 which claims priority to U.S. Ser. No. 61/286,831 filed Dec. 16, 2009, U.S. Ser. No. 61/286,559 filed Dec. 15, 2009, and U.S. Ser. No. 61/405,778 filed Oct. 22, 2010, each of which are hereby incorporated by reference in their entirety.

### BACKGROUND

Systems that deliver certain drugs to a patient (e.g., targeted to a particular tissue or cell type or targeted to a specific diseased tissue but not normal tissue), or that control release of drugs has long been recognized as beneficial. For example, therapeutics that include an active drug and that are capable of locating in a particular tissue or cell type e.g., a specific diseased tissue, may reduce the amount of the drug in tissues of the body that do not require treatment. This is particularly important when treating a condition such as cancer where it is desirable that a cytotoxic dose of the drug is delivered to cancer cells without killing the surrounding non-cancerous tissue. Further, such therapeutics may reduce the undesirable and sometimes life threatening side effects common in anticancer therapy. For example, nanoparticle therapeutics may, due the small size, evade recognition within the body allowing for targeted and controlled delivery while e.g., remaining stable for an effective amount of time.

Therapeutics that offer such therapy and/or controlled release and/or targeted therapy also must be able to deliver an effective amount of drug. It can be a challenge to prepare nanoparticle systems that have an appropriate amount of drug associated each nanoparticle, while keeping the size of the nanoparticles small enough to have advantageous delivery properties. For example, while it is desirable to load a nanoparticle with a high quantity of therapeutic agent, nanoparticle preparations that use a drug load that is too high will result in nanoparticles that are too large for practical therapeutic use. Further, it may be desirable for therapeutic nanoparticles to remain stable so as to e.g. substantially limit rapid or immediate release of the therapeutic agent.

Accordingly, a need exists for new nanoparticle formulations and methods of making such nanoparticles and compositions, that can deliver therapeutic levels of drugs to treat diseases such as cancer, while also reducing patient side effects.

### SUMMARY

In one aspect, the invention provides therapeutic nanoparticle that includes an active agent or therapeutic agent, e.g. a corticosteroid such as budesonide or pharmaceutically acceptable salts thereof, and one, two, or three biocompatible polymers. For example, disclosed herein is a therapeutic nanoparticle comprising about 0.1 to about 50 weight percent of a corticosteroid (for example budesonide) and about 50 to about 99 weight percent of a biocompatible polymer, e.g., about 70 to about 99 weight percent of a biocompatible polymer. For example, the biocompatible polymer may be a diblock poly(lactic) acid-poly(ethylene)glycol copolymer (e.g. PLA-PEG) or a diblock (poly(lactic)-co-poly (glycolic) acid)-poly(ethylene)glycol copolymer (e.g. PLGA-PEG), or the biocompatible polymer may include two or more different biocompatible polymers, for example, the therapeutic nanoparticles can also include a homopolymer such as a poly(lactic) acid homopolymer. For example, a disclosed therapeutic nanoparticle may include about 0.1 to about 50 weight percent, or about 1 to about 20 weight percent of a corticosteroid; and about 50 to about 99 weight percent, or about 70 to about 99 weight percent biocompatible polymer, wherein the biocompatible polymer is selected from the group consisting of a) a diblock poly(lactic) acid-poly(ethylene)glycol copolymer, b) a diblock poly(lactic)-co-poly (glycolic) acid-poly(ethylene)glycol copolymer, c) a combination of a) and a poly (lactic) acid homopolymer; d) a combination of b) and a poly (lactic) acid homopolymer; e) 1,2 distearoyl-sn-glycero-3-phosphoethanolamine-poly(ethylene)glycol copolymer; and f) a combination of e) and a poly (lactic) acid homopolymer or poly(lactic)-co-(glycolic) acid.

The diameter of disclosed nanoparticles may be, for example, about 60 to about 230 nm, about 60 to about 120 nm, about 70 to about 120 nm, about 70 to about 140 nm or about 80 to about 130 nm.

In another embodiment, disclosed particles may substantially release less than about 20%, or less than about 40%, or less than about 50%, or even less than about 60% of the corticosteroid when placed in a phosphate buffer solution at room temperature, or at 37° C.

Corticosteroids may include for example, budesonide, fluocinonide, triamcinolone, mometasone, amcinonide, halcinonide, ciclesonide, beclomethasone, or a pharmaceutically acceptable salt thereof. For example, contemplated nanoparticles may include about 1 to about 16 weight percent of a corticosteroid. In another example, contemplated nanoparticles may include about 1 to about 9 weight percent of a corticosteroid, Disclosed therapeutic nanoparticles may include about 1 to about 12 weight percent budesonide.

For example, disclosed nanoparticles may include a biocompatible polymer that is a diblock poly(lactic) acid-poly(ethylene)glycol copolymer. Diblock poly(lactic) acid-poly(ethylene)glycol copolymers that may form part of a disclosed nanoparticle may comprise poly(lactic acid) having a number average molecular weight of about 15 to 20 kDa and poly(ethylene)glycol having a number average molecular weight of about 4 to about 6 kDa. Diblock poly(lactic)-co-glycolic acid-poly(ethylene)glycol copolymer may include poly(lactic acid)-co-glycolic acid having a number average molecular weight of about 15 to 20 kDa, e.g., about 16 kDa and poly(ethylene)glycol having a number average molecular weight of about 4 to about 6 kDa, about 5 kDa. The poly(lactic)-co-poly (glycolic) acid portion of a contemplated diblock poly(lactic)-co-poly (glycolic) acid-poly(ethylene)glycol copolymer may have, in certain embodiments, about 50 mole percent glycolic acid and about 50 mole percent poly(lactic) acid.

An exemplary therapeutic nanoparticle may include about 40 to about 50 weight percent diblock poly(lactic)acid-poly(ethylene)glycol copolymer and about 40 to about 49, or about 40 to about 60 weight percent poly (lactic) acid homopolymer. Such poly (lactic) acid homopolymers may have e.g., a weight average molecular weight of about 15 to about 130 kDa, e.g., about 10 kDa.

In an optional embodiment, a disclosed nanoparticle may further include about 0.2 to about 10 weight percent of a diblock poly(lactic)-co-poly (glycolic) acid-poly(ethylene)glycol copolymer covalently bound to a targeting ligand.

Also disclosed herein is a pharmaceutically acceptable composition comprising a plurality of disclosed therapeutic nanoparticles and a pharmaceutically acceptable excipient. Exemplary pharmaceutically acceptable excipients may include a sugar such as sucrose.

Also disclosed herein are methods of treating asthma, osteoarthritis, dermatitis, inflammatory bowel disease, Crohn's disease, or ulcerative colitis, comprising administering an effective amount of disclosed nanoparticles.

In another embodiment, provided herein is plurality of therapeutic nanoparticles prepared by combining corticosteroids or pharmaceutically acceptable salts thereof and a diblock poly(lactic)acid-polyethylene glycol or a diblock poly(lactic)acid-co-poly(glycolic)acid-polyethylene glycol polymer and optionally a homopolymer, with an organic solvent to form a first organic phase having about 10 to about 80% weight percent solids, or about 20 to about 70 weight percent solids; combining the first organic phase with a first aqueous solution to form a coarse emulsion; emulsifying the coarse emulsion to form an emulsion phase; quenching the emulsion phase to form a quenched phase; adding a drug solubilizer to the quenched phase to form a solubilized phase of unencapsulated therapeutic agent; and filtering the solubilized phase to recover the nanoparticles, thereby forming a slurry of therapeutic nanoparticles each having about 0.1 to about 50 weight percent of a corticosteroid.

### BRIEF DESCRIPTION OF THE DRAWINGS

FIG. 1 is flow chart for an emulsion process for forming disclosed nanoparticle.

FIG. 2 is a flow diagram for a disclosed emulsion process.

FIG. 3 depicts drug load of prepared nanoparticles as a function of quench: emulsion (Q:E) ratio.

FIG. 4 depicts the in vitro release of budesonide of various nanoparticles disclosed herein.

FIG. 5 depicts the in vitro release of budesonide of various nanoparticles disclosed herein.

FIG. 6 depicts pharmacokinetics of budesonide and budesonide nanoparticles following a singe intravenous dose (0.5 mg/kg).

FIG. 7 indicates disease scores in rat intestines in a model of IBD after treatment with budesonide, budesonide PTNP and dexamethasone.

FIG. 8 indicates rat intestinal weights in a Model of IBD after Treatment with budesonide, budesonide PTNP and dexamethasone.

FIG. 9 depicts the in vitro release of budesonide in various nanoparticles.

FIG. 10 depicts the in vitro release of ciclesonide in various nanoparticles.

FIG. 11 depicts the in vitro release of beclomethasone dipropionate in various nanoparticles.

FIG. 12 depicts the in vitro release of mometasone furoate in various nanoparticles.

### DETAILED DESCRIPTION

The present invention generally relates to polymeric nanoparticles that include an active or therapeutic agent or drug, and methods of making and using such therapeutic nanoparticles. In general, a “nanoparticle” refers to any particle having a diameter of less than 1000 nm, e.g. about 10 nm to about 200 nm. Disclosed therapeutic nanoparticles may include nanoparticles having a diameter of about 60 to about 230 nm, or about 60 to about 120 nm, or about 70 to about 130 nm, or about 60 to about 140 nm, or about 70 nm to about 140 nm.

Disclosed nanoparticles may include about about 0.1 to about 50 weight percent, about 0.2 to about 35 weight percent, about 3 to about 40 weight percent, about 5 to about 30 weight percent, about 1 to about 20 weight percent, about 10 to about 30 weight percent, about 15 to 25 weight percent, or even about 4 to about 25 weight percent of an active agent, such as corticosteroid, e.g. budesonide.

Nanoparticles disclosed herein include one, two, three or more biocompatible and/or biodegradable polymers. For example, a contemplated nanoparticle may include about 50 to about 99 weight percent of one, two, three or more biocompatible polymers such as one or more co-polymers (e.g. a diblock polymer) that includes a biodegradable polymer (for example poly(lactic)acid and polyethylene glycol) and optionally about 0 to about 50 weight percent of a homopolymer, e.g. biodegradable polymer such as poly(lactic) acid.

### Polymers

In some embodiments, disclosed nanoparticles include a matrix of polymers. Disclosed nanoparticles may include one or more polymers, e.g. a diblock co-polymer and/or a monopolymer. Disclosed therapeutic nanoparticles may include a therapeutic agent that can be associated with the surface of, encapsulated within, surrounded by, and/or dispersed throughout a polymeric matrix.

A wide variety of polymers and methods for forming particles therefrom are known in the art of drug delivery. In some embodiments, the disclosure is directed toward nanoparticles with at least one polymer, for example, a first polymer that may be a co-polymer, e.g. a diblock co-polymer, and optionally a polymer that may be for example a homopolymer.

Any polymer can be used in accordance with the present invention. Polymers can be natural or unnatural (synthetic) polymers. Polymers can be homopolymers or copolymers comprising two or more monomers. In terms of sequence, copolymers can be random, block, or comprise a combination of random and block sequences. Contemplated polymers may be biocompatible and/or biodegradable.

The term “polymer,” as used herein, is given its ordinary meaning as used in the art, i.e., a molecular structure comprising one or more repeat units (monomers), connected by covalent bonds. The repeat units may all be identical, or in some cases, there may be more than one type of repeat unit present within the polymer. In some cases, the polymer can be biologically derived, i.e., a biopolymer. Non-limiting examples include peptides or proteins. In some cases, additional moieties may also be present in the polymer, for example biological moieties such as those described below. If more than one type of repeat unit is present within the polymer, then the polymer is said to be a “copolymer.” It is to be understood that in any embodiment employing a polymer, the polymer being employed may be a copolymer in some cases. The repeat units forming the copolymer may be arranged in any fashion. For example, the repeat units may be arranged in a random order, in an alternating order, or as a block copolymer, i.e., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

Disclosed particles can include copolymers, which, in some embodiments, describes two or more polymers (such as those described herein) that have been associated with each other, usually by covalent bonding of the two or more polymers together. Thus, a copolymer may comprise a first polymer and a second polymer, which have been conjugated together to form a block copolymer where the first polymer can be a first block of the block copolymer and the second polymer can be a second block of the block copolymer. Of course, those of ordinary skill in the art will understand that a block copolymer may, in some cases, contain multiple blocks of polymer, and that a “block copolymer,” as used herein, is not limited to only block copolymers having only a single first block and a single second block. For instance, a block copolymer may comprise a first block comprising a first polymer, a second block comprising a second polymer, and a third block comprising a third polymer or the first polymer, etc. In some cases, block copolymers can contain any number of first blocks of a first polymer and second blocks of a second polymer (and in certain cases, third blocks, fourth blocks, etc.). In addition, it should be noted that block copolymers can also be formed, in some instances, from other block copolymers. For example, a first block copolymer may be conjugated to another polymer (which may be a homopolymer, a biopolymer, another block copolymer, etc.), to form a new block copolymer containing multiple types of blocks, and/or to other moieties (e.g., to non-polymeric moieties).

In some embodiments, the polymer (e.g., copolymer, e.g., block copolymer) can be amphiphilic, i.e., having a hydrophilic portion and a hydrophobic portion, or a relatively hydrophilic portion and a relatively hydrophobic portion. A hydrophilic polymer can be one generally that attracts water and a hydrophobic polymer can be one that generally repels water. A hydrophilic or a hydrophobic polymer can be identified, for example, by preparing a sample of the polymer and measuring its contact angle with water (typically, the polymer will have a contact angle of less than 60°, while a hydrophobic polymer will have a contact angle of greater than about 60°). In some cases, the hydrophilicity of two or more polymers may be measured relative to each other, i.e., a first polymer may be more hydrophilic than a second polymer. For instance, the first polymer may have a smaller contact angle than the second polymer.

In one set of embodiments, a polymer (e.g., copolymer, e.g., block copolymer) contemplated herein includes a biocompatible polymer, i.e., the polymer that does not typically induce an adverse response when inserted or injected into a living subject, for example, without significant inflammation and/or acute rejection of the polymer by the immune system, for instance, via a T-cell response. Accordingly, the therapeutic particles contemplated herein can be non-immunogenic. The term non-immunogenic as used herein refers to endogenous growth factor in its native state which normally elicits no, or only minimal levels of, circulating antibodies, T-cells, or reactive immune cells, and which normally does not elicit in the individual an immune response against itself.

Biocompatibility typically refers to the acute rejection of material by at least a portion of the immune system, i.e., a nonbiocompatible material implanted into a subject provokes an immune response in the subject that can be severe enough such that the rejection of the material by the immune system cannot be adequately controlled, and often is of a degree such that the material must be removed from the subject. One simple test to determine biocompatibility can be to expose a polymer to cells in vitro; biocompatible polymers are polymers that typically will not result in significant cell death at moderate concentrations, e.g., at concentrations of 50 micrograms/10⁶ cells. For instance, a biocompatible polymer may cause less than about 20% cell death when exposed to cells such as fibroblasts or epithelial cells, even if phagocytosed or otherwise uptaken by such cells. Non-limiting examples of biocompatible polymers that may be useful in various embodiments of the present invention include polydioxanone (PDO), polyhydroxyalkanoate, polyhydroxybutyrate, poly(glycerol sebacate), polyglycolide, polylactide, PLGA, polycaprolactone, or copolymers or derivatives including these and/or other polymers.

In certain embodiments, contemplated biocompatible polymers may be biodegradable, i.e., the polymer is able to degrade, chemically and/or biologically, within a physiological environment, such as within the body. As used herein, “biodegradable” polymers are those that, when introduced into cells, are broken down by the cellular machinery (biologically degradable) and/or by a chemical process, such as hydrolysis, (chemically degradable) into components that the cells can either reuse or dispose of without significant toxic effect on the cells. In one embodiment, the biodegradable polymer and their degradation byproducts can be biocompatible.

For instance, a contemplated polymer may be one that hydrolyzes spontaneously upon exposure to water (e.g., within a subject), the polymer may degrade upon exposure to heat (e.g., at temperatures of about 37° C.). Degradation of a polymer may occur at varying rates, depending on the polymer or copolymer used. For example, the half-life of the polymer (the time at which 50% of the polymer can be degraded into monomers and/or other nonpolymeric moieties) may be on the order of days, weeks, months, or years, depending on the polymer. The polymers may be biologically degraded, e.g., by enzymatic activity or cellular machinery, in some cases, for example, through exposure to a lysozyme (e.g., having relatively low pH). In some cases, the polymers may be broken down into monomers and/or other nonpolymeric moieties that cells can either reuse or dispose of without significant toxic effect on the cells (for example, polylactide may be hydrolyzed to form lactic acid, polyglycolide may be hydrolyzed to form glycolic acid, etc.).

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEGylated polymers and copolymers of lactide and glycolide (e.g., PEGylated PLA, PEGylated PGA, PEGylated PLGA, and derivatives thereof. In some embodiments, polyesters include, for example, polyanhydrides, poly(ortho ester) PEGylated poly(ortho ester), poly(caprolactone), PEGylated poly(caprolactone), polylysine, PEGylated polylysine, poly(ethylene imine), PEGylated poly(ethylene imine), poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA can be characterized by the ratio of lactic acid:glycolic acid. Lactic acid can be L-lactic acid, D-lactic acid, or D,L-lactic acid. The degradation rate of PLGA can be adjusted by altering the lactic acid-glycolic acid ratio. In some embodiments, PLGA to be used in accordance with the present invention can be characterized by a lactic acid:glycolic acid molar ratio of approximately 85:15, approximately 75:25, approximately 60:40, approximately 50:50, approximately 40:60, approximately 25:75, or approximately 15:85.

In some embodiments, the ratio of lactic acid to glycolic acid monomers in the polymer of the particle (e.g., the PLGA block copolymer or PLGA-PEG block copolymer), may be selected to optimize for various parameters such as water uptake, therapeutic agent release and/or polymer degradation kinetics can be optimized.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), methacrylic acid alkylamide copolymer, poly(methyl methacrylate), poly(methacrylic acid polyacrylamide, amino alkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids (e.g. DNA, RNA, or derivatives thereof). Amine-containing polymers such as poly(lysine), polyethylene imine (PEI), and poly(amidoamine) dendrimers are contemplated for use, in some embodiments, in a disclosed particle.

In some embodiments, polymers can be degradable polyesters bearing cationic side chains. Examples of these polyesters include poly(L-lactide-co-L-lysine), poly(serine ester), and poly(4-hydroxy-L-proline ester). A polymer (e.g., copolymer, e.g., block copolymer) containing poly(ethylene glycol) repeat units can also be referred to as a “PEGylated” polymer. Such polymers can control inflammation and/or immunogenicity (i.e., the ability to provoke an immune response) and/or lower the rate of clearance from the circulatory system via the reticuloendothelial system (RES), due to the presence of the poly(ethylene glycol) groups.

PEGylation may also be used, in some cases, to decrease charge interaction between a polymer and a biological moiety, e.g., by creating a hydrophilic layer on the surface of the polymer, which may shield the polymer from interacting with the biological moiety. In some cases, the addition of poly(ethylene glycol) repeat units may increase plasma half-life of the polymer (e.g., copolymer, e.g., block copolymer), for instance, by decreasing the uptake of the polymer by the phagocytic system while decreasing transfection/uptake efficiency by cells. Those of ordinary skill in the art will know of methods and techniques for PEGylating a polymer, for example, by using EDC (1-ethyl-3-(3-dimethylaminopropyl) carbodiimide hydrochloride) and NHS (N-hydroxysuccinimide) to react a polymer to a PEG group terminating in an amine, by ring opening polymerization techniques (ROMP), or the like.

It is contemplated that PEG may include a terminal end group, for example, when PEG is not conjugated to a ligand. For example, PEG may terminate in a hydroxyl, a methoxy or other alkoxyl group, a methyl or other alkyl group, an aryl group, a carboxylic acid, an amine, an amide, an acetyl group, a guanidino group, or an imidazole. Other contemplated end groups include azide, alkyne, maleimide, aldehyde, hydrazide, hydroxylamine, alkoxyamine, or thiol moieties.

Particles disclosed herein may or may not contain PEG. In addition, certain embodiments can be directed towards copolymers containing poly(ester-ether)s, e.g., polymers having repeat units joined by ester bonds (e.g., R—C(O)—O—R′ bonds) and ether bonds (e.g., R—O—R′ bonds). In some embodiments of the invention, a biodegradable polymer, such as a hydrolyzable polymer, containing carboxylic acid groups, may be conjugated with poly(ethylene glycol) repeat units to form a poly(ester-ether).

In one embodiment, the molecular weight of the polymers can be optimized for effective treatment as disclosed herein. For example, the molecular weight of a polymer may influence particle degradation rate (such as when the molecular weight of a biodegradable polymer can be adjusted), solubility, water uptake, and drug release kinetics. For example, the molecular weight of the polymer can be adjusted such that the particle biodegrades in the subject being treated within a reasonable period of time (ranging from a few hours to 1-2 weeks, 3-4 weeks, 5-6 weeks, 7-8 weeks, etc.). A disclosed particle can for example comprise a copolymer of PEG and PLA, the PEG can have a molecular weight of 1,000-20,000 Da, e.g., 5,000-20,000 Da, e.g., 10,000-20,000 Da, and the PLA or PLA can have a molecular weight of 5,000-100,000 Da, e.g., 20,000-70,000 Da, e.g., 15,000-50,000 Da.

For example, disclosed here is an exemplary therapeutic nanoparticle that includes about 10 to about 99 weight percent poly(lactic) acid-poly(ethylene)glycol copolymer or poly(lactic)-co-poly (glycolic) acid-poly(ethylene)glycol copolymer, or about 20 to about 80 weight percent, about 40 to about 80 weight percent, or about 30 to about 50 weight percent, or about 70 to about 90 weight percent poly(lactic) acid-poly(ethylene)glycol copolymer or poly(lactic)-co-poly (glycolic) acid-poly(ethylene)glycol copolymer. Exemplary poly(lactic) acid-poly(ethylene)glycol copolymers can include a number average molecular weight of about 15 to about 20 kDa, or about 10 to about 25 kDa of poly(lactic) acid and a number average molecular weight of about 4 to about 6, or about 2 kDa to about 10 kDa of poly(ethylene)glycol.

Disclosed nanoparticles may optionally include about 1 to about 50 weight percent poly(lactic) acid or poly(lactic) acid-co-poly (glycolic) acid (which does not include PEG, e.g a homopolymer of PLA), or may optionally include about 1 to about 50 weight percent, or about 10 to about 50 weight percent or about 30 to about 50 weight percent poly(lactic) acid or poly(lactic) acid-co-poly (glycolic) acid. In an embodiment, disclosed nanoparticles may include two polymers, e.g. PLA-PEG and PLA, in a weight ratio of about 40:60 to about 60: 40, e.g, about 50:50.

Such substantially homopolymeric poly(lactic) or poly(lactic)-co-poly(glycolic) acid may have a weight average molecular weight of about 10 to about 130 kDa, for example, about 20 to about 30 kDa, or about 100 to about 130 kDa. Such homopolymeric PLA may have a number average molecule weight of about 5 to about 90 kDa, or about 5 to about 12 kDa, about 15 to about 30 kDa, or about 60 to about 90 kDa. Exemplary homopolymeric PLA may have a number average molecular weight of about 80 kDa or a weight average molecular weight of about 124 kD. As is known in the art, molecular weight of polymers can be related to an inherent viscosity. In some embodiments, homopolymer PLA may have an inherent viscosity of about 0.2 to about 0.4, e.g. about 0.4; in other embodiments, PLA may have an inherent viscosity of about 0.6 to about 0.8. Exemplary PLGA may have a number average molecular weight of about 8 to about 12 kDa.

In certain embodiments, disclosed polymers of may be conjugated to a lipid, e.g. “end-capped,” for example, may include a lipid-terminated PEG. As described below, the lipid portion of the polymer can be used for self assembly with another polymer, facilitating the formation of a nanoparticle. For example, a hydrophilic polymer could be conjugated to a lipid that will self assemble with a hydrophobic polymer.

Exemplary lipids include fatty acids such as long chain (e.g., C₈-C₅₀), substituted or unsubstituted hydrocarbons. In some embodiments, a fatty acid group can be a C₁₀-C₂₀ fatty acid or salt thereof. In some embodiments, a fatty acid group can be a C₁₅-C₂₀ fatty acid or salt thereof. In some embodiments, a fatty acid can be unsaturated, monounsaturated, or polyunsaturated. For example, a fatty acid group can be one or more of butyric, caproic, caprylic, capric, lauric, myristic, palmitic, stearic, arachidic, behenic, or lignoceric acid. In some embodiments, a fatty acid group can be one or more of palmitoleic, oleic, vaccenic, linoleic, alpha-linolenic, gamma-linoleic, arachidonic, gadoleic, arachidonic, eicosapentaenoic, docosahexaenoic, or erucic acid.

In a particular embodiment, the lipid is of the Formula V:

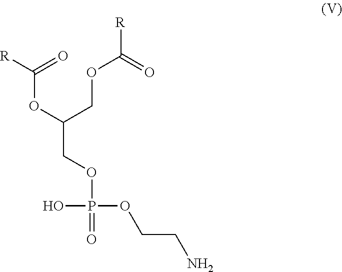

and salts thereof, wherein each R is, independently, C₁₋₃₀ alkyl. In one embodiment of Formula V, the lipid is 1,2 distearoyl-sn-glycero-3-phosphoethanolamine (DSPE), and salts thereof, e.g., the sodium salt, for example, DSPE may be conjugated to PEG via the —NH moiety.

In one embodiment, optional small molecule targeting moieties are bonded, e.g., covalently bonded, to the lipid component of the nanoparticle. For example, contemplated herein is also a nanoparticle comprising a therapeutic agent, a polymeric matrix comprising functionalized and non-functionalized polymers, a lipid, and a low-molecular weight targeting ligand, wherein the targeting ligand is bonded, e.g., covalently bonded, to the lipid component of the nanoparticle.

### Targeting Moieties

Provided herein are nanoparticles that may include an optional targeting moiety, i.e., a moiety able to bind to or otherwise associate with a biological entity, for example, a membrane component, a cell surface receptor, prostate specific membrane antigen, or the like. A targeting moiety present on the surface of the particle may allow the particle to become localized at a particular targeting site, for instance, a tumor, a disease site, a tissue, an organ, a type of cell, etc. The drug or other payload may then, in some cases, be released from the particle and allowed to interact locally with the particular targeting site.

In one embodiment of the instant invention, the targeting moiety may be a low-molecular weight ligand, e.g., a low-molecular weight PSMA ligand. For example, a targeting portion may cause the particles to become localized to a tumor, a disease site, a tissue, an organ, a type of cell, etc. within the body of a subject, depending on the targeting moiety used. For example, a low-molecular weight PSMA ligand may become localized to prostate cancer cells. The subject may be a human or non-human animal. Examples of subjects include, but are not limited to, a mammal such as a dog, a cat, a horse, a donkey, a rabbit, a cow, a pig, a sheep, a goat, a rat, a mouse, a guinea pig, a hamster, a primate, a human or the like.

Contemplated targeting moieties include small molecules. In certain embodiments, the term “small molecule” refers to organic compounds, whether naturally-occurring or artificially created (e.g., via chemical synthesis) that have relatively low molecular weight and that are not proteins, polypeptides, or nucleic acids. Small molecules typically have multiple carbon-carbon bonds. In certain embodiments, small molecules are less than about 2000 g/mol in size. In some embodiments, small molecules are less than about 1500 g/mol or less than about 1000 g/mol. In some embodiments, small molecules are less than about 800 g/mol or less than about 500 g/mol, for example about 100 g/mol to about 600 g/mol, or about 200 g/mol to about 500 g/mol. For example, a ligand may be a the low-molecular weight PSMA ligand such as

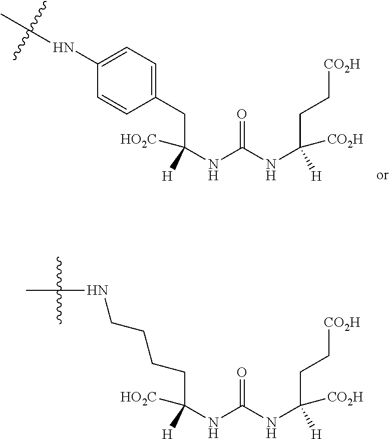 [SMILES: *Nc1ccc(C[C@H](NC(=O)N[C@@H](CCC(=O)O)C(=O)O)C(=O)O)cc1 ; "or" ; *NCCCC[C@H](NC(=O)N[C@@H](CCC(=O)O)C(=O)O)C(=O)O]

and enantiomers, stereoisomers, rotamers, tautomers, diastereomers, or racemates thereof.

In some embodiments, small molecule targeting moieties that may be used to target cells associated with prostate cancer tumors include PSMA peptidase inhibitors such as 2-PMPA, GPI5232, VA-033, phenylalkylphosphonamidates and/or analogs and derivatives thereof. In some embodiments, small molecule targeting moieties that may be used to target cells associated with prostate cancer tumors include thiol and indole thiol derivatives, such as 2-MPPA and 3-(2-mercaptoethyl)-1H-indole-2-carboxylic acid derivatives. In some embodiments, small molecule targeting moieties that may be used to target cells associated with prostate cancer tumors include hydroxamate derivatives. In some embodiments, small molecule targeting moieties that may be used to target cells associated with prostate cancer tumors include PBDA- and urea-based inhibitors, such as ZJ 43, ZJ 11, ZJ 17, ZJ 38 and/or and analogs and derivatives thereof, androgen receptor targeting agents (ARTAs), polyamines, such as putrescine, spermine, and spermidine, inhibitors of the enzyme glutamate carboxylase II (GCPII), also known as NAAG Peptidase or NAALADase.

In another embodiment of the instant invention, the targeting moiety can be a ligand that targets Her2, EGFR, or toll receptors. For example, contemplated the targeting moieties may include a nucleic acid, polypeptide, glycoprotein, carbohydrate, or lipid. For example, a targeting moiety can be a nucleic acid targeting moiety (e.g. an aptamer, e.g., the A10 aptamer) that binds to a cell type specific marker. In general, an aptamer is an oligonucleotide (e.g., DNA, RNA, or an analog or derivative thereof) that binds to a particular target, such as a polypeptide. In some embodiments, a targeting moiety may be a naturally occurring or synthetic ligand for a cell surface receptor, e.g., a growth factor, hormone, LDL, transferrin, etc. A targeting moiety can be an antibody, which term is intended to include antibody fragments, characteristic portions of antibodies, single chain targeting moieties can be identified, e.g., using procedures such as phage display. Targeting moieties may be a targeting peptide or targeting peptidomimetic has a length of up to about 50 residues. For example, targeting moieties may include the amino acid sequence AKERC, CREKA, ARYLQKLN or AXYLZZLN, wherein X and Z are variable amino acids, or conservative variants or peptidomimetics thereof. In particular embodiments, the targeting moiety is a peptide that includes the amino acid sequence AKERC, CREKA, ARYLQKLN or AXYLZZLN, wherein X and Z are variable amino acids, and has a length of less than 20, 50 or 100 residues. The CREKA (Cys Arg Glu Lys Ala) peptide or a peptidomimetic thereof peptide or the octapeptide AXYLZZLN are also contemplated as targeting moieties, as well as peptides, or conservative variants or peptidomimetics thereof, that binds or forms a complex with collagen IV, or the targets tissue basement membrane (e.g., the basement membrane of a blood vessel), can be used as a targeting moiety.

Exemplary targeting moieties include peptides that target ICAM (intercellular adhesion molecule, e.g. ICAM-1).

Targeting moieties disclosed herein are typically conjugated to a disclosed polymer or copolymer (e.g. PLA-PEG), and such a polymer conjugate may form part of a disclosed nanoparticle. For example, a disclosed therapeutic nanoparticle may optionally include about 0.2 to about 10 weight percent of a PLA-PEG or PLGA-PEG, wherein the PEG is functionalized with a targeting ligand. Contemplated therapeutic nanoparticles may include, for example, about 0.2 to about 10 mole percent PLA-PEG-ligand or poly (lactic) acid -co-poly (glycolic) acid-PEG-ligand. For example, PLA-PEG-ligand may include a PLA with a number average molecular weight of about 10 kDa to about 20 kDa and PEG with a number average molecular weight of about 4,000 to about 8,000 Da.

### Nanoparticles

Disclosed nanoparticles may have a substantially spherical (i.e., the particles generally appear to be spherical), or non-spherical configuration. For instance, the particles, upon swelling or shrinkage, may adopt a non-spherical configuration. In some cases, the particles may include polymeric blends. For instance, a polymer blend may include a first co-polymer that includes polyethylene glycol and a second polymer.

Disclosed nanoparticles may have a characteristic dimension of less than about 1 micrometer, where the characteristic dimension of a particle is the diameter of a perfect sphere having the same volume as the particle. For example, the particle can have a characteristic dimension of the particle can be less than about 300 nm, less than about 200 nm, less than about 150 nm, less than about 100 nm, less than about 50 nm, less than about 30 nm, less than about 10 nm, less than about 3 nm, or less than about 1 nm in some cases. In particular embodiments, disclosed nanoparticles may have a diameter of about 60 nm to about 230 nm, about 70 nm to about 200 nm, about 70 nm to about 180 nm, about 80 nm to about 130 nm, or about 80 nm to about 120 nm.

In one set of embodiments, the particles can have an interior and a surface, where the surface has a composition different from the interior, i.e., there may be at least one compound present in the interior but not present on the surface (or vice versa), and/or at least one compound is present in the interior and on the surface at differing concentrations. For example, in one embodiment, a compound, such as a targeting moiety (i.e., a low-molecular weight ligand) of a polymeric conjugate of the present invention, may be present in both the interior and the surface of the particle, but at a higher concentration on the surface than in the interior of the particle, although in some cases, the concentration in the interior of the particle may be essentially nonzero, i.e., there is a detectable amount of the compound present in the interior of the particle.

In some cases, the interior of the particle is more hydrophobic than the surface of the particle. For instance, the interior of the particle may be relatively hydrophobic with respect to the surface of the particle, and a drug or other payload may be hydrophobic, and readily associates with the relatively hydrophobic center of the particle. The drug or other payload can thus be contained within the interior of the particle, which can shelter it from the external environment surrounding the particle (or vice versa). For instance, a drug or other payload contained within a particle administered to a subject will be protected from a subject's body, and the body may also be substantially isolated from the drug for at least a period of time.

For example, disclosed herein is a therapeutic polymeric nanoparticle comprising a first non-functionalized polymer; an optional second non-functionalized polymer; an optional functionalized polymer comprising a targeting moiety; and a therapeutic agent. In a particular embodiment, the first non-functionalized polymer is PLA, PLGA, or PEG, or copolymers thereof, e.g. a diblock co-polymer PLA-PEG. For example, exemplary nanoparticle may have a PEG corona with a density of about 0.065 g/cm³, or about 0.01 to about 0.10 g/cm³.

Disclosed nanoparticles may be stable, for example in a solution that may contain a saccharide, e.g. sugar, for at least about 3 days, at least about 4 days or at least about 5 days at room temperature, or at 25° C.

In some embodiments, disclosed nanoparticles may also include a fatty alcohol, which may increase the rate of drug release. For example, disclosed nanoparticles may include a C₈-C₃₀ alcohol such as cetyl alcohol, octanol, stearyl alcohol, arachidyl alcohol, docosonal, or octasonal.

Nanoparticles may have controlled release properties, e.g., may be capable of delivering an amount of active agent to a patient, e.g., to specific site in a patient, over an extended period of time, e.g. over 1 day, 1 week, or more. In some embodiments, disclosed nanoparticles substantially immediately releases (e.g. over about 1 minute to about 30 minutes) less than about 2%, less than about 4%, less than about 5%, or less than about 10% of an active agent (e.g. budesodine), for example when placed in a phosphate buffer solution at room temperature and/or at 37° C. In an embodiment, disclosed nanoparticles substantially immediately releases less than about 20% of the therapeutic agent in less than 1 hour when placed in a phosphate buffer solution at 37° C.

In another embodiment, a disclosed nanoparticle may release less than about 20%, less than about 30%, less than about 40%, less than 50%, or even less than 60% (or more) for example when placed in a phosphate buffer solution at room temperature or at 37° C., for 2 days or more.

In one embodiment, the invention comprises a nanoparticle comprising 1) a polymeric matrix and 2) an amphiphilic compound or layer that surrounds or is dispersed within the polymeric matrix forming a continuous or discontinuous shell for the particle. An amphiphilic layer can reduce water penetration into the nanoparticle, thereby enhancing drug encapsulation efficiency and slowing drug release. Further, these amphipilic layer protected nanoparticles can provide therapeutic advantages by releasing the encapsulated drug and polymer at appropriate times.

As used herein, the term “amphiphilic” refers to a property where a molecule has both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. Exemplary amphiphilic compound include, for example, one or a plurality of the following: naturally derived lipids, surfactants, or synthesized compounds with both hydrophilic and hydrophobic moieties.

Specific examples of amphiphilic compounds include, but are not limited to, phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), most preferably between 0.1-30 (weight lipid/w polymer). Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

In a particular embodiment, an amphiphilic component may include lecithin, and/or in particular, phosphatidylcholine.

### Preparation of Nanoparticles

Another aspect of the invention is directed to systems and methods of making disclosed nanoparticles. In some embodiments, using two or more different polymers (e.g., a copolymer such as a diblock copolymer and a homopolymer) properties of particles may be controlled.

In a particular embodiment, the methods described herein form nanoparticles that have a high amount of encapsulated therapeutic agent, for example, may include about 0.1 to about 50 weight percent, or about 1 to about 40 weight percent, or about 1 to about 30 weight percent, e.g. about 10 to about 25 weight percent or about 5 to about 20 weight percent corticosteroid.

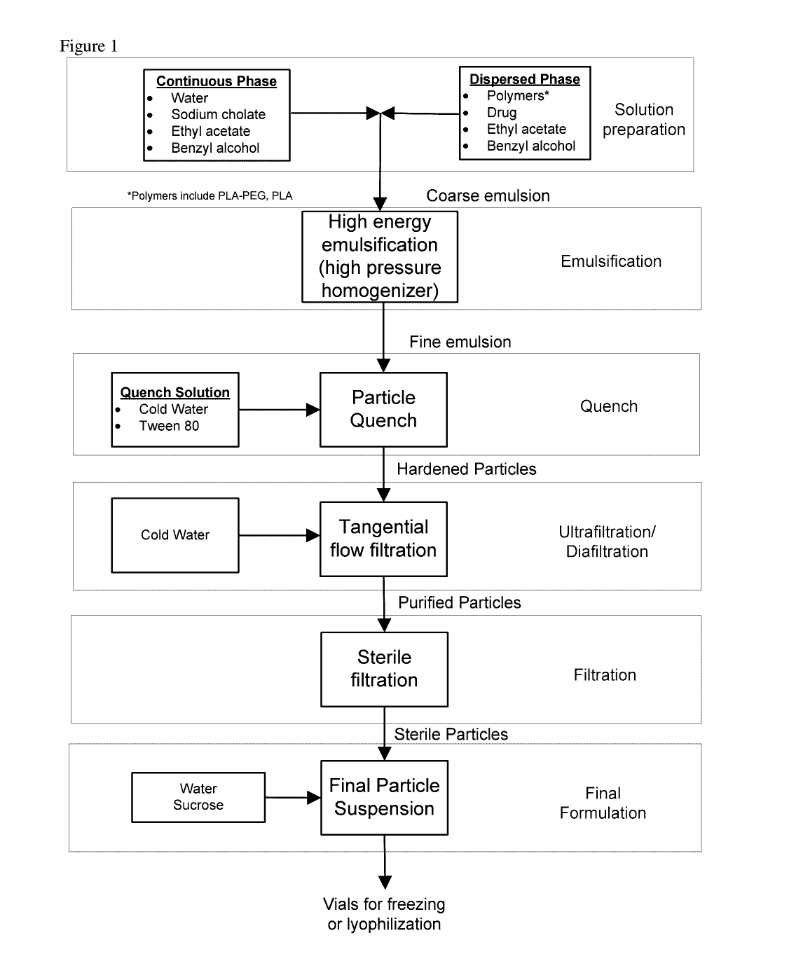

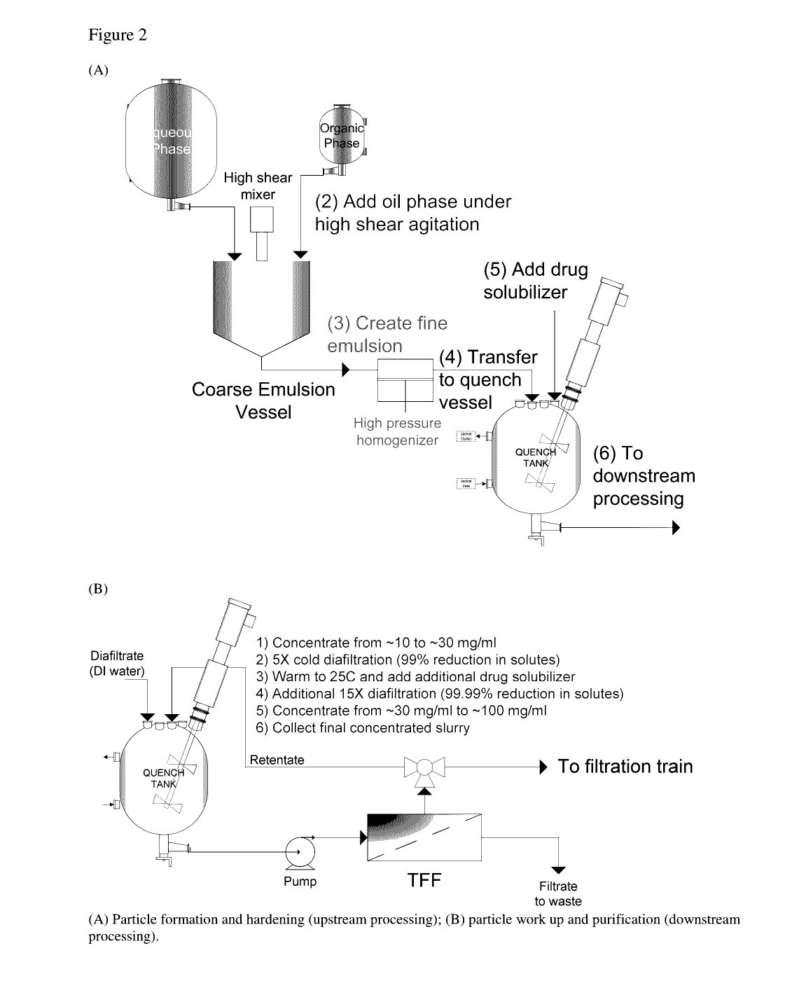

In an embodiment, a nanoemulsion process is provided, such as the process represented in FIGS. 1 and 2. For example, a therapeutic agent, a first polymer (for example, PLA-PEG or PLGA-PEG) and a second polymer (e.g. (PL(G)A or PLA), with an organic solution to form a first organic phase. Such first phase may include about 5 to about 90% weight percent solids, e.g about 5 to about 80% solids, or about 10 to about 40% solids, e.g. about 10%, 15%, 20%, 30%, 40%, 50%, 60%, 70%, or 80% weight percent solids. Solids typically refers to weight percent of polymer(s) and active. The first organic phase may be combined with a first aqueous solution to form a coarse emulsion. The organic solution can include, for example, acetonitrile, tetrahydrofuran, ethyl acetate, isopropyl alcohol, isopropyl acetate, dimethylformamide, methylene chloride, dichloromethane, chloroform, acetone, benzyl alcohol, Tween 80, Span 80, or the like, and combinations thereof. In an embodiment, the organic phase may include benzyl alcohol, ethyl acetate, and combinations thereof. The coarse emulsion can be between about 1 and 60 weight percent, e.g., 5-40 weight percent, solids. The aqueous solution can be water, optionally in combination with one or more of sodium cholate, ethyl acetate, and benzyl alcohol.

For example, the oil or organic phase may use solvent that is only partially miscible with the nonsolvent (water). Therefore, when mixed at a low enough ratio and/or when using water pre-saturated with the organic solvents, the oil phase remains liquid. The oil phase may bee emulsified into an aqueous solution and, as liquid droplets, sheared into nanoparticles using, for example, high energy dispersion systems, such as homogenizers or sonicators. The aqueous portion of the emulsion, otherwise known as the “water phase”, may be surfactant solution consisting of sodium cholate and pre-saturated with ethyl acetate and benzyl alcohol.

Emulsifying the coarse emulsion to form an emulsion phase may be performed in one or two emulsification steps. For example, a primary emulsion may be prepared, and then emulsified to form a fine emulsion. The primary emulsion can be formed, for example, using simple mixing, a high pressure homogenizer, probe sonicator, stir bar, or a rotor stator homogenizer. The primary emulsion may be formed into a fine emulsion through the use of e.g. probe sonicator or a high pressure homogenizer, e.g. by using 1, 2, 3 or more passes through a homogenizer. For example, when a high pressure homogenizer is used, the pressure used may be about 4000 to about 8000 psi, or about 4000 to about 5000 psi, e.g. 4000 or 5000 psi.

Either solvent evaporation or dilution may be needed to complete the extraction of the solvent and solidify the particles. For better control over the kinetics of extraction and a more scalable process, a solvent dilution via aqueous quench may be used. For example, the emulsion can be diluted into cold water to a concentration sufficient to dissolve all of the organic solvent to form a quenched phase. Quenching may be performed at least partially at a temperature of about 5° C. or less. For example, water used in the quenching may be at a temperature that is less that room temperature (e.g. about 0 to about 10° C., or about 0 to about 5° C.).

In some embodiments, not all of the therapeutic agent is encapsulated in the particles at this stage, and a drug solubilizer is added to the quenched phase to form a solubilized phase. The drug solubilizer may be for example, Tween 80, Tween 20, polyvinyl pyrrolidone, cyclodextran, sodium dodecyl sulfate, or sodium cholate. For example, Tween-80 may added to the quenched nanoparticle suspension to solubilize the free drug and prevent the formation of drug crystals. In some embodiments, a ratio of drug solubilizer to therapeutic agent is about 100:1 to about 10:1.

The solubilized phase may be filtered to recover the nanoparticles. For example, ultrafiltration membranes may be used to concentrate the nanoparticle suspension and substantially eliminate organic solvent, free drug, and other processing aids (surfactants). Exemplary filtration may be performed using a tangential flow filtration system. For example, by using a membrane with a pore size suitable to retain nanoparticles while allowing solutes, micelles, and organic solvent to pass, nanoparticles can be selectively separated. Exemplary membranes with molecular weight cut-offs of about 300-500 kDa (˜5-25 nm) may be used.

Diafiltration may be performed using a constant volume approach, meaning the diafiltrate (cold deionized water, e.g. about 0° C. to about 5° C., or 0 to about 10° C.) may added to the feed suspension at the same rate as the filtrate is removed from the suspension. In some embodiments, filtering may include a first filtering using a first temperature of about 0° C. to about 5° C., or 0° C. to about 10° C., and a second temperature of about 20° C. to about 30° C., or 15° C. to about 35° C. For example, filtering may include processing about 1 to about 6 diavolumes at about 0° C. to about 5° C., and processing at least one diavolume (e.g. about 1 to about 3 or about 1-2 diavolumes) at about 20° C. to about 30° C.

After purifying and concentrating the nanoparticle suspension, the particles may be passed through one, two or more sterilizing and/or depth filters, for example, using ˜0.2 μm depth pre-filter.

In exemplary embodiment of preparing nanoparticles, an organic phase is formed composed of a mixture of a corticosteroid, and polymer (homopolymer, and co-polymer). The organic phase may be mixed with an aqueous phase at approximately a 1:5 ratio (oil phase:aqueous phase) where the aqueous phase is composed of a surfactant and optionally dissolved solvent. A primary emulsion may then formed by the combination of the two phases under simple mixing or through the use of a rotor stator homogenizer. The primary emulsion is then formed into a fine emulsion through the use of e.g. high pressure homogenizer. Such fine emulsion may then quenched by, e.g. addition to deionized water under mixing. An exemplary quench:emulsion ratio may be about approximately 8.5:1. A solution of Tween (e.g., Tween 80) can then be added to the quench to achieve e.g. approximately 2% Tween overall, which may serves to dissolve free, unencapsulated drug. Formed nanoparticles may then be isolated through either centrifugation or ultrafiltration/diafiltration.

### Therapeutic Agents

According to the present invention, any agents including, for example, therapeutic agents (e.g. anti-cancer agents), diagnostic agents (e.g. contrast agents; radionuclides; and fluorescent, luminescent, and magnetic moieties), prophylactic agents (e.g. vaccines), and/or nutraceutical agents (e.g. vitamins, minerals, etc.) may be delivered by the disclosed nanoparticles. Exemplary agents to be delivered in accordance with the present invention include, but are not limited to, small molecules (e.g. cytotoxic agents), nucleic acids (e.g., siRNA, RNAi, and microRNA agents), proteins (e.g. antibodies), peptides, lipids, carbohydrates, hormones, metals, radioactive elements and compounds, drugs, vaccines, immunological agents, etc., and/or combinations thereof. In some embodiments, the agent to be delivered is an agent useful in the treatment of cancer (e.g., an anti-neoplastic agent).

In a particular embodiment, the drug may be released in a controlled release manner from the particle and allowed to interact locally with the particular patient site (e.g., a tumor). The term “controlled release” is generally meant to encompass release of a substance (e.g., a drug) at a selected site or otherwise controllable in rate, interval, and/or amount. Controlled release encompasses, but is not necessarily limited to, substantially continuous delivery, patterned delivery (e.g., intermittent delivery over a period of time that is interrupted by regular or irregular time intervals), and delivery of a bolus of a selected substance (e.g., as a predetermined, discrete amount if a substance over a relatively short period of time (e.g., a few seconds or minutes)).

The active agent or drug may be a corticosteroid such as budesonide, fluocinonide, triamcinolone, mometasone, amcinonide, halcinonide, ciclesonide, beclomethasone, or a pharmaceutically acceptable salt thereof. Other contemplated corticosteroids include: hydrocortisone, cortisone, prednisolone, methylpredinsolone, prednisone, betamethasone, dexamethasone, fluocortolone, fluprednidene, clobetasol-17-propionate, predicarbate, or pharmaceutically acceptable salts thereof.

In an embodiment, an active agent may (or may not be) conjugated to e.g. a disclosed hydrophobic polymer that forms part of a disclosed nanoparticle, e.g an active agent may be conjugated (e.g. covalently bound, e.g. directly or through a linking moiety such as linking moiety comprising —NH-alkylene-C(O)—) to PLA or PLGA, or a PLA or PLGA portion of a copolymer such as PLA-PEG or PLGA-PEG.

### Pharmaceutical Formulations

Nanoparticles disclosed herein may be combined with pharmaceutical acceptable carriers to form a pharmaceutical composition. As would be appreciated by one of skill in this art, the carriers may be chosen based on the route of administration as described below, the location of the target issue, the drug being delivered, the time course of delivery of the drug, etc.

The pharmaceutical compositions and particles disclosed herein can be administered to a patient by any means known in the art including oral and parenteral routes. The term “patient,” as used herein, refers to humans as well as non-humans, including, for example, mammals, birds, reptiles, amphibians, and fish. For instance, the non-humans may be mammals (e.g., a rodent, a mouse, a rat, a rabbit, a monkey, a dog, a cat, a primate, or a pig). In certain embodiments parenteral routes are desirable since they avoid contact with the digestive enzymes that are found in the alimentary canal. According to such embodiments, inventive compositions may be administered by injection (e.g., intravenous, subcutaneous or intramuscular, intraperitoneal injection), rectally, vaginally, topically (as by powders, creams, ointments, or drops), or by inhalation (as by sprays).

In a particular embodiment, disclosed nanoparticles may be administered to a subject in need thereof systemically, e.g., by IV infusion or injection.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In one embodiment, the inventive conjugate is suspended in a carrier fluid comprising 1% (w/v) sodium carboxymethyl cellulose and 0.1% (v/v) TWEEN™ 80. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Disclosed nanoparticles may be formulated in dosage unit form for ease of administration and uniformity of dosage. The expression “dosage unit form” as used herein refers to a physically discrete unit of nanoparticle appropriate for the patient to be treated. For any nanoparticle, the therapeutically effective dose can be estimated initially either in cell culture assays or in animal models, usually mice, rabbits, dogs, or pigs. An animal model may also used to achieve a desirable concentration range and route of administration. Such information can then be used to determine useful doses and routes for administration in humans. Therapeutic efficacy and toxicity of nanoparticles can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies can be used in formulating a range of dosage for human use.

In an exemplary embodiment, a pharmaceutical composition is disclosed that includes a plurality of nanoparticles each comprising a therapeutic agent; and a pharmaceutically acceptable excipient.

In some embodiments, a composition suitable for freezing is contemplated, including nanoparticles disclosed herein and a solution suitable for freezing, e.g., a sugar (e.g. sucrose) solution is added to a nanoparticle suspension. The sucrose may e.g., act as a cryoprotectant to prevent the particles from aggregating upon freezing. For example, provided herein is a nanoparticle formulation comprising a plurality of disclosed nanoparticles, sucrose and water; wherein, for example, the nanoparticles/sucrose/water are present at about 5-10%/10-15%/80-90% (w/w/w).

### Methods of Treatment

In some embodiments, therapeutic particles disclosed herein may be used to treat, alleviate, ameliorate, relieve, delay onset of, inhibit progression of, reduce severity of, and/or reduce incidence of one or more symptoms or features of a disease, disorder, and/or condition. For example, disclosed therapeutic particles, that include e.g., a corticosteroid such as budesonide thereof may be used to treat asthma, osteoarthritis, dermatitis, and inflammatory disorders such as inflammatory bowel disease, ulcerative colitis, and/or Crohn's disease. Treatment of cancers such as colon cancer is also contemplated herein.

Disclosed methods for the treatment of inflammatory disorders may comprise administering a therapeutically effective amount of the disclosed therapeutic particles to a subject in need thereof, in such amounts and for such time as is necessary to achieve the desired result. In certain embodiments of the present invention a “therapeutically effective amount” is that amount effective for treating, alleviating, ameliorating, relieving, delaying onset of, inhibiting progression of, reducing severity of, and/or reducing incidence of one or more symptoms or features of e.g. an inflammatory disorder being treated.

Also provided herein are therapeutic protocols that include administering a therapeutically effective amount of an disclosed therapeutic particle to a healthy individual (i.e., a subject who does not display any symptoms of an inflammatory disorder). For example, healthy individuals may be “immunized” with an inventive targeted particle prior to development of cancer and/or onset of symptoms of inflammation; at risk individuals (e.g., patients who have a family history of inflammation; patients carrying one or more genetic mutations associated with development of an inflammation disorder; patients having a genetic polymorphism associated with development of an inflammation disorder.

### EXAMPLES

The invention now being generally described, it will be more readily understood by reference to the following examples which are included merely for purposes of illustration of certain aspects and embodiments of the present invention, and are not intended to limit the invention in any way.

### Example 1

### Preparation of PLA-PEG

The synthesis is accomplished by ring opening polymerization of d,l-lactide with α-hydroxy-ω-methoxypoly(ethylene glycol) as the macro-initiator, and performed at an elevated temperature using Tin (II) 2-Ethyl hexanoate as a catalyst, as shown below (PEG Mn≈5,000 Da; PLA Mn≈16,000 Da; PEGPLA Mₙ≈21,000 Da)

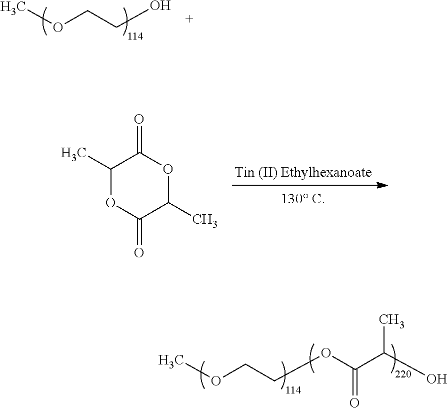

The polymer is purified by dissolving the polymer in dichloromethane, and precipitating it in a mixture of hexane and diethyl ether. The polymer recovered from this step shall be dried in an oven.

### Example 2

### Nanoparticle Preparation

All budesonide batches were produced as follows, unless noted otherwise. Drug and polymer (16/5 PLA-PEG) constituents were dissolved in the oil phase organic solvent system, typically 70% ethyl acetate (EA) and 30% benzyl alcohol (BA), at 20% or 30 wt % [solids]. The aqueous phase consisted mainly of water, pre-saturated with 2% benzyl alcohol and 4% ethyl acetate, with sodium cholate (SC) as surfactant. The coarse O/W emulsion was prepared by dumping the oil phase into the aqueous phase under rotor stator homogenization at an oil: aqueous ratio of 1:5 or 1:10. The fine emulsion was then prepared by processing the coarse emulsion through a Microfluidics high pressure homogenizer (generally M110S pneumatic) at 9000 psi through a 100 μm Z-interaction chamber. The emulsion was then quenched into a cold DI water quench at 10:1 or 5:1 quench: emulsion ratio. Polysorbate 80 (Tween 80) was then added as a process solubilizer to solubilize the unencapsulated drug. The batch was then processed with ultrafiltration followed by diafiltration to remove solvents, unencapsulated drug and solubilizer. This process is depicted pictorially in FIGS. 1 and 2.

The particle size measurements were performed by Brookhaven DLS and/or Horiba laser diffraction. To determine drug load, slurry samples were submitted for HPLC and [solids] analysis. The slurry retains were then diluted with sucrose to 10% before freezing. All ratios listed are on a w/w basis, unless specified otherwise.

Tween 80 may be used post quench to remove unencapsulated drug.

### Example 3

### In Vitro Release

An in vitro release method is used to determine the initial burst phase release from nanoparticles at both ambient and 37° C. conditions. Nanoparticles are placed into sink conditions for the API and mixed in a water bath. Released and encapsulated drug are separated by using an ultracentrifuge.

The dialysis system can be as follows: 3 mL slurry of budesonide nanoparticles (approx 250 μg/mL budesonide PLGA/PLA nanoparticles, corresponding to 2.5 mg/mL solid concentration) in DI-water is placed into the inner tube of a 300 kDa MWCO dialyzer by pipetting. The nanoparticle is suspended in this media. The dialyzer is placed into a glass bottles containing 130 ml release media (2.5% hydroxyl beta cyclodextrin in PBS), which is continually stirred at 150 rpm using a shaker to prevent the formation of an unstirred water layer at the membrane/outer solution interface. At pre-determined time points, aliquot of samples (1 mL) were withdrawn from the outer solution (dialysate) and analyzed for budesonide concentration by HPLC.

Alternatively, nanoparticles are placed into sink conditions for the API and mixed in a water bath. Released and encapsulated drug are separated by using an ultracentrifuge.

The centrifugal system is run as follows: 3 mL slurry of budesonide nanoparticles (approx 250 μg/mL budesonide PLGA/PLA nanoparticles) in DI-water is placed into glass bottles containing 130 ml release media (2.5% hydroxyl beta cyclodextrin in 1×PBS), which is continually stirred at 150 rpm using a shaker. At pre-determined time points, aliquot of samples (4 mL) were withdrawn. Samples are centrifuged at 236,000 g for 60 minutes and the supernatant is assayed for budesonide content to measured released budesonide.

### Example 4

### Particle Size Analysis

Particle size is analyzed by two techniques—dynamic light scattering (DLS) and laser diffraction. DLS is performed using a Brookhaven ZetaPals instrument at 25° C. in dilute aqueous suspension using a 660 nm laser scattered at 90°. Laser diffraction is performed with a Horiba LS950 instrument in dilute aqueous suspension using both a HeNe laser at 633 nm and an LED at 405 nm, scattered at 90° and analyzed using the Mie optical model. Alternatively, particle size is analyzed using the Accusizer SPOS.

### Example 5

Nanoparticles with various drug loads were prepared by varying the following parameters: vary Q:E ratio (5:1, 15:1 and 30:1); increase [solids] to 30% by reducing initial budesonide to 10%; increase particle size by reducing surfactant to 0.5%.

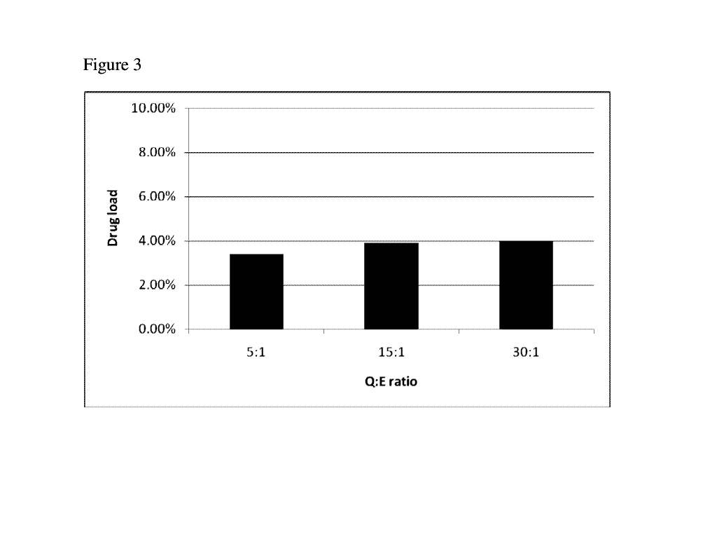

A single emulsion was made at 30% solids, 10% drug, and emulsion was split into three different quenches at Q:E ratios of 5:1, 15:1 and 30:1. The particle size was 137 nm and drug load ranged from 3.4% to 4%. FIG. 3 depicts the drug load of the nanoparticles as a function of Q:E ratio. The increased drug load may be due to increased [solids] and particle size, while varying Q:E ratio did not seem to have a significant effect on drug load.

A 10 g batch was made for scale up using the formulation and process of Example 2, using 30% solids and 10% Microfluidics M110EH electric high pressure homogenizer was used to make this batch at 900 psi using a 200 um Z-chamber. Particle size was 113 nm and drug load was 3.8% (Batch 55-40, control).

### Example 6

### Nanoparticles

Various batches on nanoparticles were prepared using the general procedure of Example 2, and using the following parameters:

16/5 PLA-PEG with mid MW PLA (IV (inherent viscosity)=0.3) at 40% solids: Batch #52-198

16/5 PLA-PEG with 40%[solids]; 10% drug, using 60/40 of ethyl acetate/benzyl alcohol Batch: #58-27-1

16/5 PLA-PEG with 40% [solids] and 5% [drug]: Batch #58-27-2

16/5 PLA-PEG with high MW PLA (IV=0.6-0.8) at 40% solids: Batch #41-171-A

High MW PLA (IV=0.6-0.8) with DSPE-PEG (2 k): Batch #41-171-B & 61-8-B

16/5 PLA-PEG with high MW PLA (IV=0.6-0.8) at 75% solids: Batch #41-176

16/5 PLA-PEG with doped high MW PLA (IV=0.6-0.8) at 75% and 50% solids: Batch #41-183-A&B

Mid MW PLA was obtained from Surmodics (also known as LakeShore (LS)), with an inherent viscosity of 0.3. 16/5 PLA-PEG was obtained from Boehringer Ingelheim (batch 41-176) or Polymer Source (batch 41-183). High MW PLA with a Mₙ of 80 kDa, M_{w} of 124 kDa was obtained from Surmodics.

| | | Size | Drug |
|---|---|---|---|
| Batch No# | Description | (nm) | Load |
| 52-198 | Doped mid MW PLA | 120 | 7.08% |
| 58-27-1 | Higher [solids] | 153 | 4.28% |
| 58-27-2 | Higher [solids] and lower initial [drug] | 101 | 1.92% |
| 41-171-A | Doped high MW PLA | 117 | 4.21% |
| 41-171-B | High MW PLA with DSPE-PEG | 224 | 6.11% |
| 41-176 | Doped high MW PLA at 75% | 181 | 5.14% |
| 41-183-A | Doped high MW PLA at 75% | 176 | 1.90% |
| 41-183-B | Doped high MW PLA at 50% | 125 | 1.72% |
| 61-8-B | High MW PLA with DSPE-PEG | 168 | 3.2% |

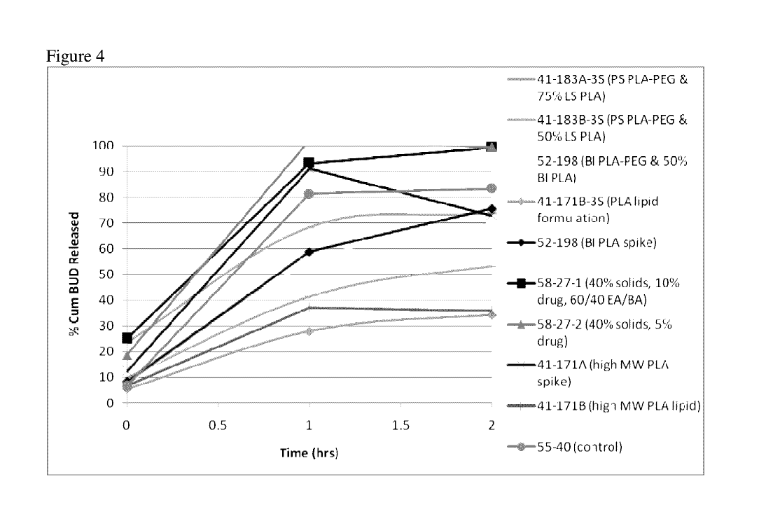

In vitro release of each batch is depicted in FIG. 4. Note: Batch 41-171-A at 1 hour time point is an outlier caused by one of the uncentrifuged samples reading extraordinarily low. Both batches 41-171-B (lipid formulation) and 41-183-A (high MW PLA) showed drug release ≦50% at 2 hours while the other formulations had released between 70-100% within 2 hours.

### Example 7

### Batch for Animal Study

A 10 g batch was made to confirm the drug load and release seen in batches 41-176 and 41-183-A as well as to provide material for animal studies. Particle size was 183 nm and drug load was 5.03%. Formulation and process parameters were scaled linearly with the exception of water phase [surfactant]. Table B below details the major differences between the batches:

TABLE B

| Parameter or attribute | 41-176 | 41-183A | 62-30 |
|---|---|---|---|
| Scale | 1 g | 1 g | 10 g |
| 16/5 Polymer supply | Boehringer | Polymer | Boehringer |
| | Ingelheim | Source | Ingelheim |
| Sodium cholate | 2% | 5% | 2.5% |
| Homogenizer | M110S | M110S | M110EH |
| Drug load | 5.14% | 1.73% | 5.03% |
| PSD | 181 | 176 | 183 |

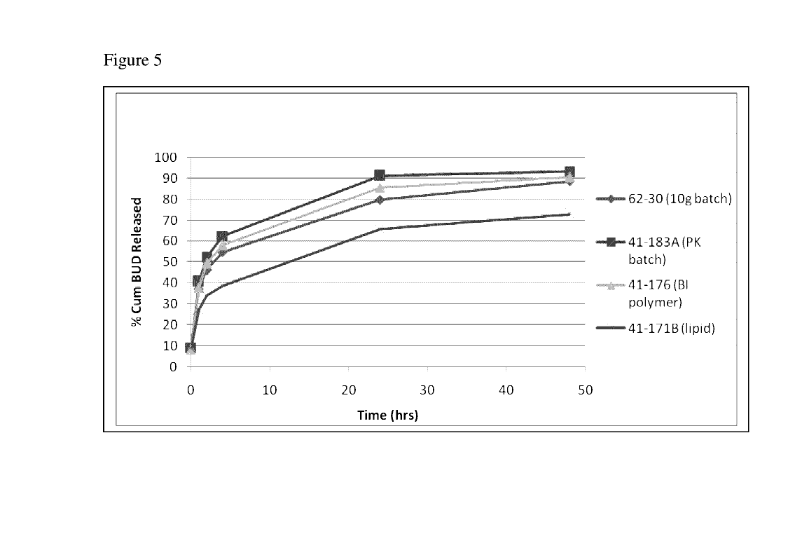

The 10 g batch, batch no. 62-30, was chosen for the PK study and was first tested for drug release to ensure the release was similar to 41-176 and 41-183-A, as shown in FIG. 5.

### Example 8

### Rat Study: Pharmacokinetics

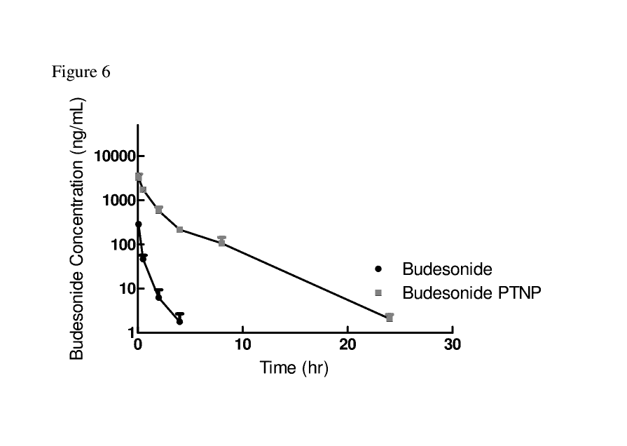

Rats (male Sprague Dawley, approximately 300 g with jugular cannulae) were given a single intravenous dose of 0.5 mg/kg of budesonide or passively targeted nanoparticles (PTNP) encapsulating budesonide (prepared as in Example 7) at time=0. At various times after dosing, blood samples were collected from the jugular cannulae into tubes containing lithium heparin, and plasma was prepared. Plasma levels were determined by extraction of the budesonide from plasma followed by LCMS analysis. The results from this PK study are shown in FIG. 6.

Encapsulation of budesonide in co-polymer nanoparticles resulted in an 11-fold increase in the maximum plasma concentration (Cmax), a 4-fold increase in half-life (t_{1/2}) and a 36-fold increase in the area under the concentration—time curve (AUC). Budesonide encapsulation also reduces the volume of distribution (Vz) by 9-fold and reduces the clearance from plasma (Cl) by 37-fold. Each of these parameter changes indicates that nanoparticle encapsulation of budesonide promotes plasma localization of budesonide at the expense of tissue distribution of the steroid. Table C outlines the pharmacokinetic analyses of budesonide and budesonide PTNP.

TABLE C
Group 1: Budesonide

| | Rat 1-1 | Rat 1-2 | Rat 1-3 | Rat 1-4 | Rat 1-5 | Rat 1-6 | Avg | sd |
|---|---|---|---|---|---|---|---|---|
| Cmax (ng/mL) | 324 | 241 | 226 | 337 | 306 | 279 | 286 | 45 |
| t_{1/2} (hr) | 0.73 | 0.75 | 0.74 | 0.69 | 0.70 | 0.87 | 0.75 | 0.06 |
| AUC_{inf} (hr * ng/mL) | 159 | 126 | 108 | 161 | 159 | 173 | 148 | 25 |
| Vz (mL/kg) | 3311 | 4291 | 4937 | 3104 | 3191 | 3627 | 3744 | 727 |
| Cl (mL/hr/kg) | 3136 | 3966 | 4611 | 3104 | 3151 | 2892 | 3477 | 668 |

Group 2: Budesonide PTNP

| | Rat 2-1 | Rat 2-2 | Rat 2-3 | Rat 2-4 | Rat 2-5 | Rat 2-6 | Avg | sd |
|---|---|---|---|---|---|---|---|---|
| Cmax (ng/mL) | 3180 | 3750 | 4140 | 3330 | 2800 | 2260 | 3243 | 669 |
| t_{1/2} (hr) | 2.66 | 2.95 | 3.18 | 3.00 | 2.98 | 2.8 | 2.9 | 0.2 |
| AUC_{inf} (hr * ng/mL) | 5614 | 6666 | 5760 | 4686 | 4299 | 5245 | 5378 | 839 |
| Vz (mL/kg) | 341 | 319 | 398 | 463 | 501 | 383 | 400.8 | 70.0 |
| Cl (mL/hr/kg) | 89 | 75 | 87 | 107 | 116 | 95 | 94.8 | 14.7 |

### Example 9

### Rat Model of Inflammatory Disease

Budesonide and Budesonide PTNP were compared in a model of inflammatory bowel disease (IBD) as an efficacy model of inflammation. In this model, female rats were given two subcutaneous doses of 8 mg/kg indomethacin at 24 hour intervals to induce lesions resembling those occurring in Crohn's disease in the small intestine. Intravenous daily treatment with vehicle, budesonide (0.02, 0.2 or 2 mg/kg) or budesonide PTNP (0.02, 0.2 or 2 mg/kg) or oral daily treatment with Dexamethasone (0.1 mg/kg) was initiated one day before indomethacin treatment (day −1) and continued for 5 total days (days −1 to 3). Animals were euthanized on day 4, and a 10 cm area at risk in the small intestine was scored for gross pathology and weighed.

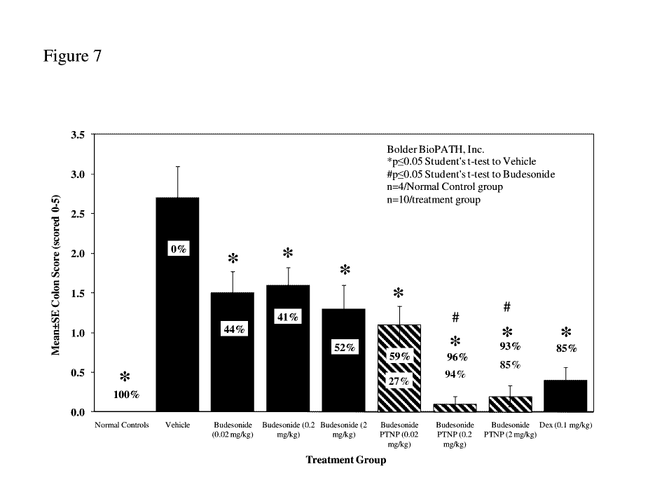

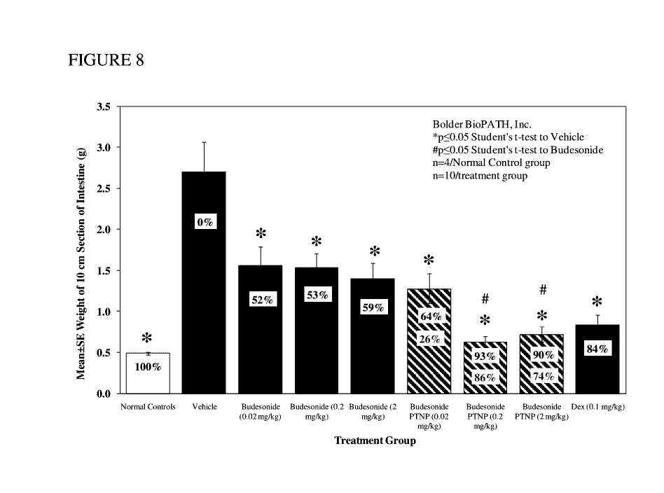

Using a disease scoring system in which a score of 0 is normal, and a score of 5 indicates death due to IBD symptoms, normal rats have an average score of 0, and an average intestinal weight of 0.488 g. In contrast, vehicle treated controls with indomethacin-induced IBD had an average clinical score of 2.7 (FIG. 7) and intestinal weight of 2.702 g (FIG. 8). Intestinal scores were significantly decreased towards normal after treatment with budesonide at doses of 0.02 mg/kg (52% decrease), 0.2 mg/kg (53% decrease) and 2 mg/kg (59% decrease; FIG. 7). In the same way, intestinal scores were significantly decreased towards normal after treatment with budesonide PTNP at doses of 0.02 mg/kg (59% decrease), 0.2 mg/kg (96% decrease) and 2 mg/kg (93% decrease; FIG. 7). Small intestine scores were also significantly decreased by treatment with 0.2 mg/kg budesonide PTNP (94%) or 2 mg/kg budesonide PTNP (85%) compared to animals treated with the same dose of budesonide free-drug (FIG. 7).

Small intestine weights were significantly decreased toward normal following treatment with budesonide at doses of 0.02 mg/kg (52% decrease), 0.2 mg/kg (53% decrease) or 2 mg/kg (59% decrease; FIG. 8). In the same way, intestinal weights were significantly decreased towards normal after treatment with budesonide PTNP at doses of 0.02 mg/kg (64% decrease), 0.2 mg/kg (93% decrease) and 2 mg/kg (90% decrease; FIG. 8). Small intestine weights were also significantly decreased by treatment with 0.2 mg/kg budesonide PTNP (86%) or 2 mg/kg budesonide PTNP (74%) compared to animals treated with the same dose of budesonide free-drug (FIG. 7). Results of this study indicate that daily intravenous treatment with budesonide or budesonide PTNP significantly inhibited the clinical parameters associated with indomethacin induced inflammatory bodwl disease in rats, with budesonide PTNP treatment having a significant beneficial effect over treatment with budesonide at corresponding dose levels.

### Example 10

### Particles with Alternate Co-Polymers

Following the general procedure of Example 2, nanoparticles were formed from budesonide with PLA-PEG co-polymers as follows:

50/5 PLA-PEG: (PLA Mw=50; PEG Mw=5); Batch #55-106-A

50/5 PLA-PEG and high MW (75 Mn PLA: Batch #55-106-B

80/10 PLA-PEG: Batch #55-106-A

80/10 PLA-PEG and high MW PLA: 55-106-B

Batches B and D had high MW 75 Mn PLA doped at 50% of total polymer.

| Batch # | Description | Drug Load |
|---|---|---|
| 55-106-A | 50/5 PLA-PEG | 2.30% |
| 55-106-B | 50/5 PLA-PEG with high MW PLA doped | 3.10% |
| 55-106-C | 80/10 PLA-PEG | 1.40% |
| 55-106-D | 80/10 PLA-PEG with high MW PLA doped | 1.50% |

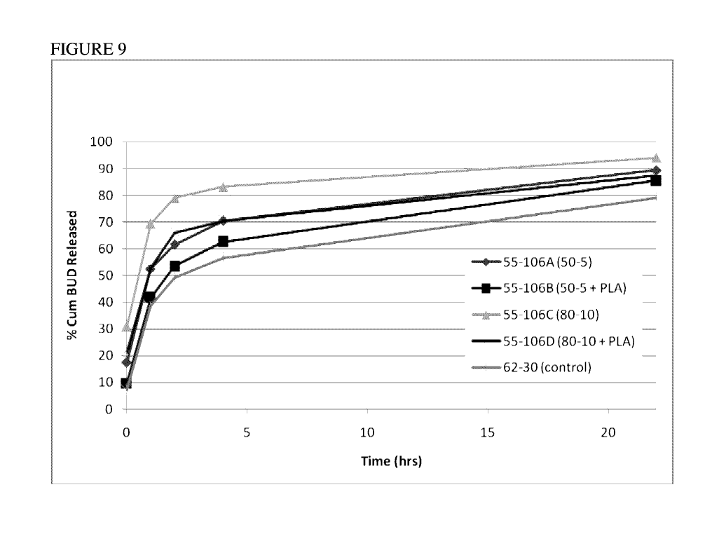

A drug release study was performed to see whether changing copolymer MW had an effect on slowing down drug release. FIG. 9 shows the release for batch #55-106-B, i.e. 50/5 PLA-PEG doped with high MW PLA, and batch 62-30.

### Example 11

### Ciclesonide Polymeric Nanoparticles

All ciclesonide batches were produced as follows, unless noted otherwise. Drug and polymer (16/5 PLA-PEG) ingredients were dissolved in the oil phase organic solvent system, typically 80% ethyl acetate (EA) and 20% benzyl alcohol (BA), at 30 wt % [solids]. The aqueous phase consisted mainly of water, pre-saturated with 2% benzyl alcohol and 4% ethyl acetate, with sodium cholate (SC) as surfactant. The coarse O/W emulsion was prepared by dumping the oil phase into the aqueous phase under rotor stator homogenization at an oil:aqueous ratio of 1:5. The fine emulsion was then prepared by processing the coarse emulsion through a Microfluidics high pressure homogenizer (generally M110S pneumatic) at 9000 psi through a 100 μm Z-interaction chamber. The emulsion was then quenched into a cold DI water quench at 10:1 quench:emulsion ratio. Polysorbate 80 (Tween 80) was then added as a process solubilizer to solubilize the unencapsulated drug at 100:1 Tween 80:drug ratio. The batch was then processed with ultrafiltration followed by diafiltration to remove solvents, unencapsulated drug, and solubilizer. The particle size measurements were performed by Brookhaven DLS. To determine drug load, slurry samples were submitted for HPLC and [solids] analysis. The slurry retains were then diluted with sucrose to 10% sucrose before freezing. All ratios listed are on a w/w basis, unless specified otherwise.

An exemplary formulation had initial solids and drug concentrations at 30% and 20% respectively. Two 2 g batches were made to evaluate drug loading and the effect of adding Tween 80 (T80) (100×) pre/post quenching. Particle size for these batches was between 88-91 nm while drug loads ranged from 6.1% to 7.5%. It was observed that in general, addition of Tween 80 post quench resulted in higher drug loads, as shown in Table E, while preventing drug crystal formation. Therefore, a 100:1 Tween 80:ciclesonide ratio was shown to be effective in removing unencapsulated drug.

TABLE E

| | | | | | Particle |
|---|---|---|---|---|---|
| Batch | | | | Drug | Size |
| 55-152- | Description | [solids] | [drug] | Load | (nm) |
| A | Ciclesonide nanoparticles, | 30% | 20% | 6.1% | 88.2 |
| | 100x T80 added pre quench | | | | |
| B | Ciclesonide nanoparticles, | 30% | 20% | 7.5% | 91 |
| | 100x T80 added post | | | | |
| | quench | | | | |

Another exemplary formulation was made in two 5 g batches. One had 16/5 PLA-PEG as the sole polymer constituent, whereas the other had 16/5 PLA-PEG with 80 k Mₙ PLA doped at 50% of total polymer. Initial solids and drug concentrations were 30% and 20%, respectively. The batches were made and processed as described in the general process above, with the exception of the PLA doped polymer batch being homogenized for 4 passes to obtain the desired particle size. Tween 80 was used as a process solubilizer at 100:1 Tween 80:drug ratio added post quench. The particle drug load ranged from 8% to 11.4%, while particle size was between 80.3 and 113.5 nm, as shown in Table F.

TABLE F

| | | | | | Particle |
|---|---|---|---|---|---|
| Batch | | | | Drug | Size |
| 55-160- | Description | [solids] | [drug] | Load | (nm) |
| A | Ciclesonide | 30% | 20% | 11.4% | 80.3 |
| | nanoparticles, 16/5 | | | | |
| | PLA-PEG | | | | |
| B | Ciclesonide | 30% | 20% | 8.1% | 113.5 |
| | nanoparticles, 16/5 | | | | |
| | PLA-PEG with 80k | | | | |
| | Mn PLA | | | | |

For in vitro release studies using the procedure of Example 3, beta cyclodextrin (BCD) was evaluated as a solubilizer for maintaining sink conditions during the release of ciclesonide into the release medium. At 10% wt, BCD was able to solubilize ciclesonide at concentrations above the required 100 μg/mL, as shown in Table G.

TABLE G

| Batch | | [ciclesonide] |
|---|---|---|
| 55-169- | Description | (μg/mL) |
| C-1 | 2.5% wt BCD | 17.58 |
| C-2 | 10% wt BCD | 133 |

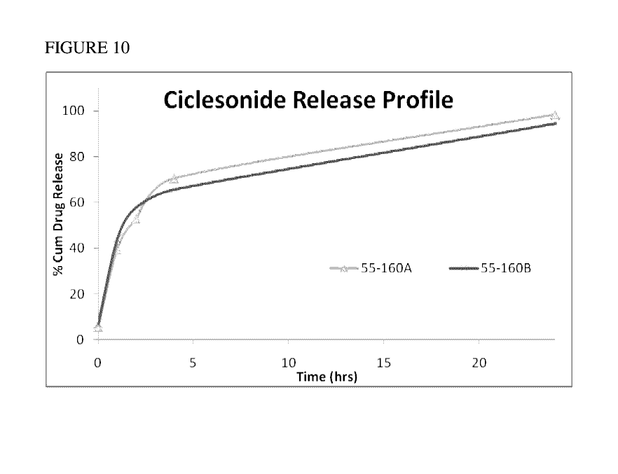

An in vitro drug release study was performed for the 55-160 batches at 37° C. 10% wt BCD was used as the in vitro solubilizer. An acetonitrile gradient beginning at 40% or lower was used for the HPLC assay method. It was observed that both formulations released about ˜50-60% ciclesonide within 2 hours and ˜100% ciclesonide within 24 hours, as shown in FIG. 10. In addition, the release profiles between PLA doped and no PLA batches were similar.

### Example 12

### Beclomethasone Dipropionate (BEC) Polymeric Nanoparticles

All BEC batches were produced as follows, unless noted otherwise. Drug and polymer (16/5 PLA-PEG) ingredients were dissolved in the oil phase organic solvent system, typically 70% ethyl acetate (EA) and 30% benzyl alcohol (BA), at 30 wt % [solids] and 10 wt % drug. The aqueous phase consisted mainly of water, pre-saturated with 2% benzyl alcohol and 4% ethyl acetate, with sodium cholate (SC) as surfactant. The coarse O/W emulsion was prepared by dumping the oil phase into the aqueous phase under rotor stator homogenization at oil:aqueous ratio of 1:5. The fine emulsion was then prepared by processing the coarse emulsion through a Microfluidics high pressure homogenizer (generally M110S pneumatic) at 9000 psi through a 100 μm Z-interaction chamber. The emulsion was then quenched into a cold DI water quench at 10:1 quench:emulsion ratio. Polysorbate 80 (Tween 80) was then added as a process solubilizer to solubilize the unencapsulated drug at 100:1 Tween 80:drug ratio. The batch was then processed with ultrafiltration followed by diafiltration to remove solvents, unencapsulated drug, and solubilizer. The particle size measurements were performed by Brookhaven DLS. To determine drug load, slurry samples were submitted for HPLC and [solids] analysis. The slurry retains were then diluted with sucrose to 10% sucrose before freezing. All ratios listed are on a w/w basis, unless specified otherwise.

An exemplary formulation had 16/5 PLA-PEG as the polymer, with initial solids and drug concentrations at 30% and 10% respectively. Two 2 g batches were made to evaluate drug loading and the effect of adding Tween 80 (T80) (100×) pre/post quenching. Particle size for these batches was between 80-82 nm while drug loads ranged from 4.8% to 5.6%, as shown in Table H. It was observed that although addition of Tween 80 post quench resulted in slightly lower loads, the drug load was within desirable range and drug crystal formation was prevented. Therefore, a 100:1 Tween 80:BEC ratio was shown to be effective in removing unencapsulated drug.

TABLE H

| | | | | | Particle |
|---|---|---|---|---|---|
| Batch | | | | Drug | Size |
| 55-149- | Description | [solids] | [drug] | Load | (nm) |
| A | BEC nanoparticles, | 30% | 10% | 5.6% | 82.8 |
| | 100x T80 added pre quench | | | | |
| B | BEC nanoparticles, | 30% | 10% | 4.8% | 80 |
| | 100x T80 added post | | | | |
| | quench | | | | |

Another exemplary formulation was made in two 5 g batches. One had 16/5 PLA-PEG as the sole polymer constituent, whereas the other had 16/5 PLA-PEG with 80 k Mₙ PLA doped at 50% of total polymer. Initial solids and drug concentrations were 30% and 10%, respectively. The batches were made and processed as described in the general process above. Tween 80 was used as a process solubilizer at 100:1 Tween 80:drug ratio added post quench. The drug load ranged from 4.9% to 6.6%, while particle size was between 79.5 and 156.5 nm, as shown in Table I.

TABLE I

| | | | | | Particle |
|---|---|---|---|---|---|
| Batch | | | | Drug | Size |
| 55-155- | Description | [solids] | [drug] | Load | (nm) |
| A | BEC nanoparticles, 16/5 | 30% | 10% | 4.9% | 79.5 |
| | PLA-PEG | | | | |
| B | BEC nanoparticles, 16/5 | 30% | 10% | 6.6% | 156.5 |
| | PLA-PEG with 80k Mn | | | | |
| | PLA | | | | |

For in vitro release studies using the procedure of Example 3, beta cyclodextrin (BCD) was evaluated as a solubilizer for maintaining sink conditions during the release of ciclesonide into the release medium. At 10% wt, BCD was able to solubilize BEC at concentrations above the required sink conditions, as shown in Table J.

TABLE J

| Batch | | [BEC] |
|---|---|---|
| 55-169- | Description | (μg/mL) |
| B-1 | 2.5% wt BCD | 9.54 |
| B-2 | 10% wt BCD | 54.6 |

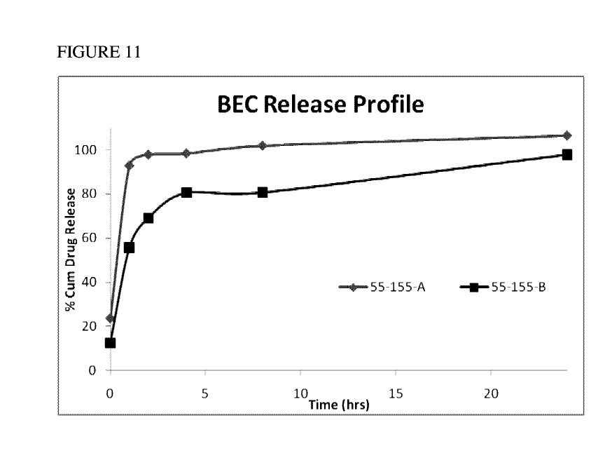

An in vitro drug release study was performed for the 55-155 batches at 37° C. 10% wt BCD was used as the in vitro solubilizer. An acetonitrile gradient beginning at 40% or lower was used for the HPLC assay method. It was observed that the 55-155A (no PLA dope) formulation released about ˜100% BEC within 2 hours, while the 55-155B (PLA dope) formulation released ˜100% BEC within 24 hours, as shown in FIG. 11.

### Example 13

### Mometasone Furoate (MOM) Polymeric Nanoparticles

All MOM batches were produced as follows, unless noted otherwise. Drug and polymer (16/5 PLA-PEG) ingredients were dissolved in the oil phase organic solvent system, typically dichloromethane (DCM), at 30 wt % [solids] and 10 wt % drug. The aqueous phase consisted mainly of water with 2 wt % sodium cholate (SC) as surfactant. The coarse O/W emulsion was prepared by dumping the oil phase into the aqueous phase under rotor stator homogenization at oil:aqueous ratio of 1:5. The fine emulsion was then prepared by processing the coarse emulsion through a Microfluidics high pressure homogenizer (generally M110S pneumatic) at 9000 psi through a 100 μm Z-interaction chamber for 6 passes. The emulsion was then quenched into a cold DI water quench at 10:1 quench:emulsion ratio. Polysorbate 80 (Tween 80) was then added as a process solubilizer and stirred over 20 minutes to solubilize the unencapsulated drug at 100:1 Tween 80:drug ratio. The batch was then processed with ultrafiltration followed by diafiltration to remove solvents, unencapsulated drug, and solubilizer. The particle size measurements were performed by Brookhaven DLS. To determine drug load, slurry samples were submitted for HPLC and [solids] analysis. The slurry retains were then diluted with sucrose to 10% sucrose before freezing. All ratios listed are on a w/w basis, unless specified otherwise.

An exemplary formulation was made in two 5 g batches. One had 16/5 PLA-PEG as the sole polymer constituent, whereas the other had 16/5 PLA-PEG with 80 k Mₙ PLA doped at 50% of total polymer. The batches were made and processed as described in the general process above. The drug load ranged from 2.7% to 6.3%, while particle size was between 83.9 and 208.4 nm, as shown in Table K.

TABLE K

| | | | | | Particle |
|---|---|---|---|---|---|
| Batch | | | | Drug | Size |
| 55-195- | Description | [solids] | [drug] | Load | (nm) |
| A | MOM nanoparticles, | 30% | 10% | 2.7% | 83.9 |
| | 16/5 PLA-PEG | | | | |
| B | MOM nanoparticles, | 30% | 10% | 6.3% | 208.4 |
| | 16/5 PLA-PEG with | | | | |
| | 80k Mn PLA | | | | |

For in vitro release studies using the procedure of Example 3, beta cyclodextrin (BCD) was evaluated as a solubilizer for maintaining sink conditions during the release of ciclesonide into the release medium. At 10% wt, BCD was able to solubilize MOM at concentrations above the required sink conditions, as shown in Table L.

TABLE L

| Batch | | [MOM] |
|---|---|---|
| 55-184- | Description | (μg/mL) |
| B-1 | 2.5% wt BCD in PBS | 33.42 |
| B-2 | 10% wt BCD in PBS | 93.5 |

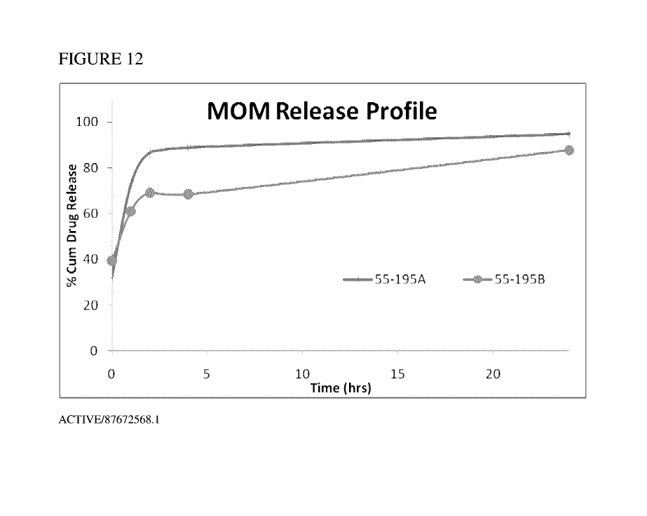

An in vitro drug release study was performed for the 55-195 batches at 37° C. 10% wt BCD was used as the in vitro solubilizer. An acetonitrile gradient beginning at 40% or lower was used for the HPLC assay method. It was observed that the 55-195A (no PLA dope) formulation released about ˜87% within 2 hours, while the 55-195B (PLA dope) formulation released ˜87% within 24 hours, as shown in FIG. 12.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications, websites, and other references cited herein are hereby expressly incorporated herein in their entireties by reference.

1. A therapeutic nanoparticle comprising:
about 0.1 to about 50 weight percent of a corticosteroid; and
about 50 to about 99 weight percent biocompatible polymer, wherein the biocompatible polymer is selected from the group consisting of
a) a diblock poly(lactic) acid-poly(ethylene)glycol copolymer;
b) a diblock poly(lactic)-co-(glycolic) acid-poly(ethylene)glycol copolymer;
c) a combination of a) and a poly (lactic) acid homopolymer or poly(lactic)-co-(glycolic) acid;
d) a combination of b) and a poly (lactic) acid homopolymer or poly(lactic)-co-(glycolic) acid;
e) 1,2 distearoyl-sn-glycero-3-phosphoethanolamine-poly(ethylene)glycol copolymer; and
f) a combination of e) and a poly (lactic) acid homopolymer or poly(lactic)-co-(glycolic) acid.

2. The therapeutic nanoparticle of claim 1 wherein said corticosteroid is selected from budesonide, fluocinonide, triamcinolone, mometasone, amcinonide, halcinonide, ciclesonide, beclomethasone, or a pharmaceutically acceptable salt thereof.

3. The therapeutic nanoparticle of claim 1, wherein the diameter of the therapeutic nanoparticle is about 60 to about 230 nm.

4. The therapeutic nanoparticle of claim 1, comprising about 1 to about 9 weight percent of the corticosteroid.

5. The therapeutic nanoparticle of claim 1, wherein said diblock poly(lactic) acid-poly(ethylene)glycol copolymer comprises poly(lactic acid) having a number average molecular weight of about 15 to 60 kDa and poly(ethylene)glycol having a number average molecular weight of about 4 to about 12 kDa.

6. The therapeutic nanoparticle of claim 1, wherein said diblock poly(lactic)-co-glycolic acid-poly(ethylene)glycol copolymer comprises poly(lactic acid)-co-glycolic acid having a number average molecular weight of about 15 to 60 kDa and poly(ethylene)glycol having a number average molecular weight of about 4 to about 12 kDa.

7. The therapeutic nanoparticle of claim 1, wherein the 1,2 distearoyl-sn-glycero-3-phosphoethanolamine-poly(ethylene)glycol copolymer comprises poly(ethylene)glycol having a number average molecular weight of about 2 kDa.

8. The therapeutic nanoparticle of claim 1, wherein the particle substantially immediately releases less than about 20% of the therapeutic agent in less than 1 hour when placed in a phosphate buffer solution at 37° C.

9. The therapeutic nanoparticle of claim 1, wherein the biocompatible polymer is diblock poly(lactic) acid-poly(ethylene)glycol copolymer.

10. The therapeutic nanoparticle of claim 1, wherein the therapeutic nanoparticle comprises about 40 to about 50 weight percent diblock poly(lactic)acid-poly(ethylene)glycol copolymer and about 40 to about 49 weight percent poly (lactic) acid homopolymer.

11. The therapeutic nanoparticle of claim 1, wherein the poly (lactic) acid homopolymer has a weight average molecular weight of about 15 to about 130 kDa.

12. The therapeutic nanoparticle of claim 1, wherein the poly (lactic) acid homopolymer has an inherent viscosity of about 0.2 to about 0.9.

13. The therapeutic nanoparticle of claim 1, wherein the poly(lactic) acid homopolymer has an inherent viscosity of about 0.4.

14. The therapeutic nanoparticle of claim 1, wherein the poly(lactic) acid homopolymer has an weight average molecular weight of about 124 kDa.

15. The therapeutic nanoparticle of claim 1, wherein said diblock poly(lactic) acid-poly(ethylene)glycol copolymer comprises poly(lactic acid) having a number average molecular weight of about 16 kDa and poly(ethylene)glycol having a number average molecular weight of about 5 kDa.

16. The therapeutic nanoparticle of claim 1, wherein said diblock poly(lactic) acid-poly(ethylene)glycol copolymer comprises poly(lactic acid) having a number average molecular weight of about 50 kDa and poly(ethylene)glycol having a number average molecular weight of about 5 kDa.

17. The therapeutic nanoparticle of claim 1, wherein said diblock poly(lactic) acid-poly(ethylene)glycol copolymer comprises poly(lactic acid) having a number average molecular weight of about 80 kDa and poly(ethylene)glycol having a number average molecular weight of about 10 kDa.

18. The therapeutic nanoparticle of claim 1, further comprising about 0.2 to about 10 weight percent of a diblock poly(lactic)-poly(ethylene)glycol copolymer covalently bound to a targeting ligand.

19. A plurality of therapeutic nanoparticles prepared by:
combining a corticosteroid or pharmaceutically acceptable salts thereof and a diblock poly(lactic)acid-polyethylene glycol or a diblock poly(lactic)acid-co-poly(glycolic)acid-polyethylene glycol polymer and optionally a poly(lactic) acid homopolymer, with an organic solvent to form a first organic phase having about 10 to about 80% solids;
combining the first organic phase with a first aqueous solution to form a coarse emulsion;
emulsifying the coarse emulstion to form an emulsion phase;
quenching the emulsion phase to form a quenched phase;
adding a drug solubilizer to the quenched phase to form a solubilized phase of unencapsulated therapeutic agent; and
filtering the solubilized phase to recover the nanoparticles, thereby forming a slurry of therapeutic nanoparticles each having about 3 to about 15 weight percent of the corticosteroid.

20. The plurality of therapeutic nanoparticles of claim 19, wherein filtering comprises concentrating, diafiltering, and terminally filtering the solubilized phase.

21. A method of treating asthma, osteoarthritis, dermatitis, inflammatory bowel disease, ulcerative colitis, or Crohn's disease comprising administering to a patient in need thereof an effective amount of a composition comprising the therapeutic nanoparticle of claim 1.

22. A controlled release therapeutic nanoparticle comprising:
about 1 to about 15 weight percent of budesonide;
a diblock polymer chosen from: poly(lactic) acid-poly(ethylene)glycol copolymer or a poly(lactic)-co-poly (glycolic) acid-poly(ethylene)glycol copolymer, wherein said therapeutic agent is released at a controlled release rate.

23. The controlled release therapeutic nanoparticle of claim 22, wherein said therapeutic agent is released over a period of at least 1 day or more when administered to a patient.
